(19) United States
(12) Patent Application Publication
(10) Pub. No.: US 2018/0280130 A1
(43) Pub. Date: Oct. 4, 2018

(54) BLOOD FLOW DIVERSION DEVICE

(71) Applicant: Saeed Ayed A. ALQAHTANI, Silver Spring, MD (US)

(72) Inventor: Saeed Ayed A. ALQAHTANI, Silver Spring, MD (US)

(21) Appl. No.: 15/474,690

(22) Filed: Mar. 30, 2017

(51) Int. Cl. A61F 2/06; A61F 2/00; A61B 17/12

(52) U.S. Cl. CPC A61F 2/06; A61F 2/0077; A61F 2002/823; A61B 17/12131; A61F 2002/065; A61B 17/12113

(57) ABSTRACT

A blood flow diversion device to treat an aneurysm at a bifurcation artery, the blood flow diversion device having a plurality of legs that anchors the blood flow diversion device onto a parent artery of the bifurcation artery, and a plurality of branches that extends from the plurality of legs. The plurality of branches includes a plurality of upstream branches positioned above the plurality of legs to lie on a upstream portion of an aneurysm entrance of the aneurysm and divert the blood flow towards children arteries of the bifurcation artery, and a plurality of downstream branches positioned above the plurality of upstream branches to extend within an internal volume of the aneurysm and promote thrombosis formation in the internal volume.

### GRANT OF NON-EXCLUSIVE RIGHT

This application was prepared with financial support from the Saudi Arabian Cultural Mission, and in consideration therefore the present inventor(s) has granted The Kingdom of Saudi Arabia a non-exclusive right to practice the present invention.

### BACKGROUND

Aneurysm formation is a leading cause of disability, death, and health care expenditure. In the United State alone, it has been estimated that about 30,000 persons suffer brain aneurysm rupture every year and ruptured brain aneurysms are fatal in about 40% of cases while about 66% of those who survive suffer some permanent neurological deficit.

To treat aneurysms conventional surgical techniques can include open brain surgery e.g. craniotomy, and placing a metal clip across the aneurysm neck.

Although such conventional surgical techniques are used as a first step for the treatment of aneurysm in some particular cases, such conventional surgical techniques present important drawbacks including complications from an open brain surgery and related infections and technical challenges.

Other conventional techniques to treat aneurysms may employ endovascular techniques that rely on fully filling the inside of the aneurysm with coils to block the blood flow. Although such conventional endovascular techniques provide a direct and more active treatment than the conventional surgical techniques and are widely used in the aneurysm treatment, such conventional endovascular techniques present important drawbacks.

Notably, these conventional endovascular techniques and coils may have a risk of rupturing the aneurysm during delivery by completely filling the aneurysm with surgical coils.

Thus, a medical device to treat aneurysms solving the aforementioned limitations effectiveness and safety is desired.

### SUMMARY

Accordingly, an object of the present disclosure is to provide a blood flow diversion device which overcomes the above-mentioned limitations of effectiveness and safety.

The blood flow diversion device of the present disclosure provides a more effective and safe technique to treat aneurysm by relying on a plurality of branches that impede blood flow and promote thrombosis formation inside the aneurysm. The plurality of branches have a palm tree shape (or flower shape) that covers an aneurysm entrance to divert blood flow away from the aneurysm and extends inside an aneurysm internal to promote blood coagulation in the aneurysm.

In one non-limiting illustrative example, a blood flow diversion device to treat an aneurysm at a bifurcation artery is presented. a plurality of legs that anchors the blood flow diversion device onto a parent artery of the bifurcation artery, a branch support affixed to the plurality of legs, a plurality of branches that extends from the branch support. The plurality of branches includes a plurality of upstream branches positioned above the plurality of legs to lie on a upstream portion of an aneurysm entrance of the aneurysm and divert the blood flow towards children arteries of the bifurcation artery, and a plurality of downstream branches positioned above the plurality of upstream branches to extend within an internal volume of the aneurysm and promote thrombosis formation in the internal volume.

In another non-limiting illustrative example, a blood flow diversion device to treat an aneurysm at a bifurcation artery is presented. a plurality of legs that anchors the blood flow diversion device onto a parent artery of the bifurcation artery, a branch support affixed to the plurality of legs, and a plurality of branches that extends from the plurality of legs to impede blood flow entering the aneurysm and promote thrombosis.

In another non-limiting illustrative example, a blood flow diversion device to treat an aneurysm at a bifurcation artery is presented. a plurality of upstream branches positioned above the plurality of legs to lie on a upstream portion of an aneurysm entrance of the aneurysm and divert the blood flow towards children arteries of the bifurcation artery, and a plurality of downstream branches positioned above the plurality of upstream branches to extend within an internal volume of the aneurysm and promote thrombosis.

### BRIEF DESCRIPTION OF THE SEVERAL VIEWS OF THE DRAWINGS

To easily identify the discussion of any particular element or act, the most significant digit or digits in a reference number refer to the figure number in which that element is first introduced.

FIG. 1 is a perspective view of a blood flow diversion device inserted in an artery bifurcation forming an aneurysm, according to certain aspects of the disclosure;

FIG. 2 is a perspective view of a plurality of legs of the blood flow diversion device, according to certain aspects of the disclosure;

FIG. 3 is a perspective view of a leg of the plurality of legs, according to certain aspects of the disclosure;

FIG. 4 is a perspective view of a plurality of branches of the blood flow diversion device, according to certain aspects of the disclosure;

FIG. 5 is a perspective view of a branch of plurality of branches, according to certain aspects of the disclosure; and

FIG. 6 is a close up view of a frame mesh of the branch, according to certain aspects of the disclosure.

### DETAILED DESCRIPTION

All publications, patent applications, patents, and other references mentioned herein are incorporated by reference in their entirety. Further, the materials, methods, and examples discussed herein are illustrative only and are not intended to be limiting.

In the drawings, like reference numerals designate identical or corresponding parts throughout the several views. Further, as used herein, the words “a”, “an”, and the like include a meaning of “one or more”, unless stated otherwise. The drawings are generally drawn not to scale unless specified otherwise or illustrating schematic structures or flowcharts.

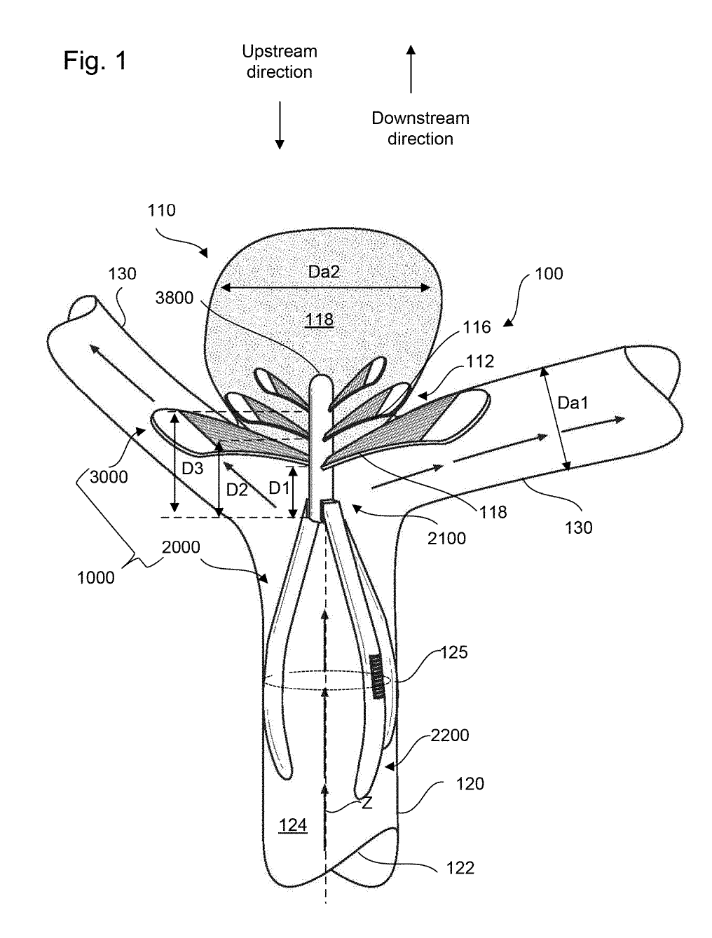

FIG. 1 is a perspective view of a blood flow diversion device 1000 inserted in an artery bifurcation 100 forming an aneurysm 110, according to certain aspects of the disclosure.

The blood flow diversion device 1000 treats the aneurysm 110 by impeding and/or slowing down blood flow coming into the aneurysm 110 from a parent artery 120 of the artery bifurcation 100 and by redirecting the blood flow towards children arteries 130 of the artery bifurcation 100. The impediment of the blood flow promotes blood coagulation and formation of thrombosis into the aneurysm 110.

The blood flow diversion device 1000 can include a plurality of legs 2000, and a plurality of branches 3000 that extends from the plurality of legs 2000. The plurality of legs 2000 can anchor the blood flow diversion device 1000 onto the parent artery 120 of the artery bifurcation 100 while the plurality of branches 3000 can be deployed around an aneurysm entrance 112 of the aneurysm 110 and inside the aneurysm 110 to impede the blood flow coming from an artery inlet 122 of the parent artery 120 and divert the blood flow towards the children arteries 130.

The anchorage of the blood flow diversion device 1000 is enhanced by an open conical shape provided by the plurality of legs 2000 while the blood flow impediment is enhanced by a palm tree shape provided by the plurality of branches 3000.

As used herein, the term “downstream” refers to the region of the blood flow diversion device 1000 closest to the aneurysm 110, and the term “upstream” refers to the region of the blood flow diversion device 1000 closest to an artery inlet 122, as illustrated by arrows in FIG. 1.

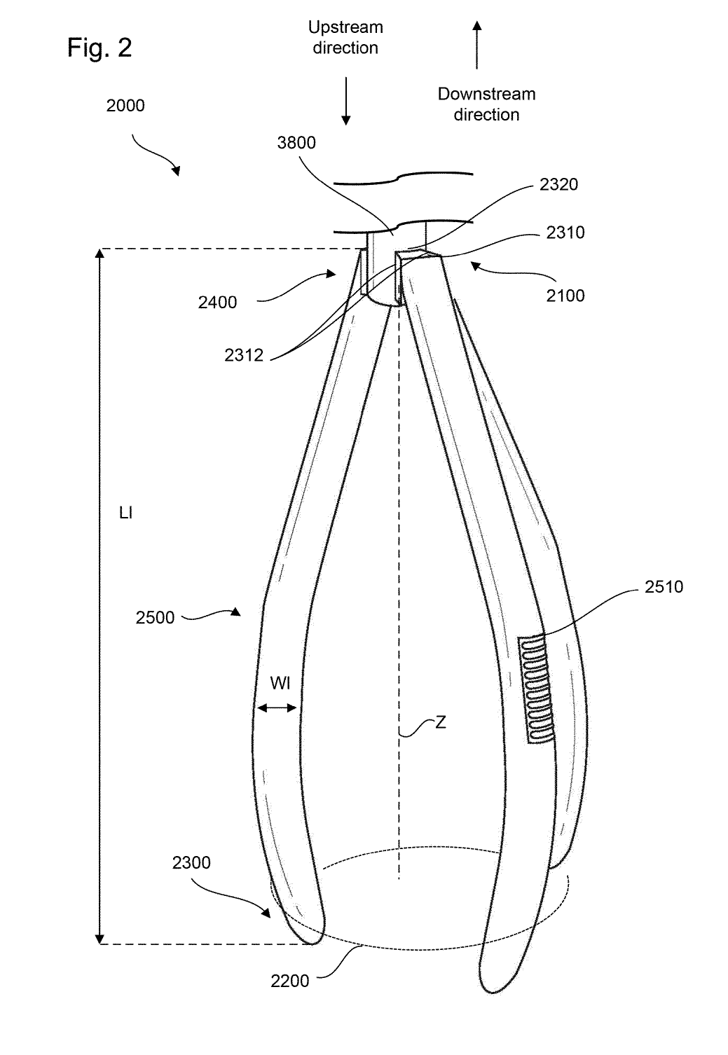

FIG. 2 is a perspective view of the plurality of legs 2000 of the blood flow diversion device 1000, according to certain aspects of the disclosure.

The plurality of legs 2000 can provide an open conical shape with a summit portion 2100 that points in the downstream direction and a base portion 2200 that points in the upstream direction.

At the summit portion 2100, the plurality of legs 2000 can be affixed onto a branch 3800 support through downstream terminal portions 2300 to support the plurality of branches 3000.

At the base portion 2200, the plurality of legs 2000 can be detached and/free from each other to independently extend from or contract towards a central axis Z that passes through the summit portion 2100 perpendicularly to the base portion 2200.

The independent extension and contraction of each leg of the plurality of legs 2000 can provide to the blood flow diversion device 1000 the ability to match different shapes that the artery internal surface 124 of the parent artery 120 can take and ensure sufficient anchorage for the blood flow diversion device 1000 for different artery morphologies.

The plurality of legs 2000 can have different configurations such as different total number of legs N1 and/or different spatial distributions. The total number of leg N1 should be sufficiently high to provide anchorage of the blood flow diversion device 1000 and sufficiently low to enable blood passage toward the children arteries 130. Similarly, the spatial distribution should be sufficiently dense to provide anchorage of the blood flow diversion device 1000 and sufficiently parse to enable blood passage towards the children arteries 130.

For example, the total number of legs N1 can be equal to three and the spatial distribution can be a uniform distribution over a circumference 125 of the artery internal surface 124 of the parent artery 120, as illustrated in FIG. 1.

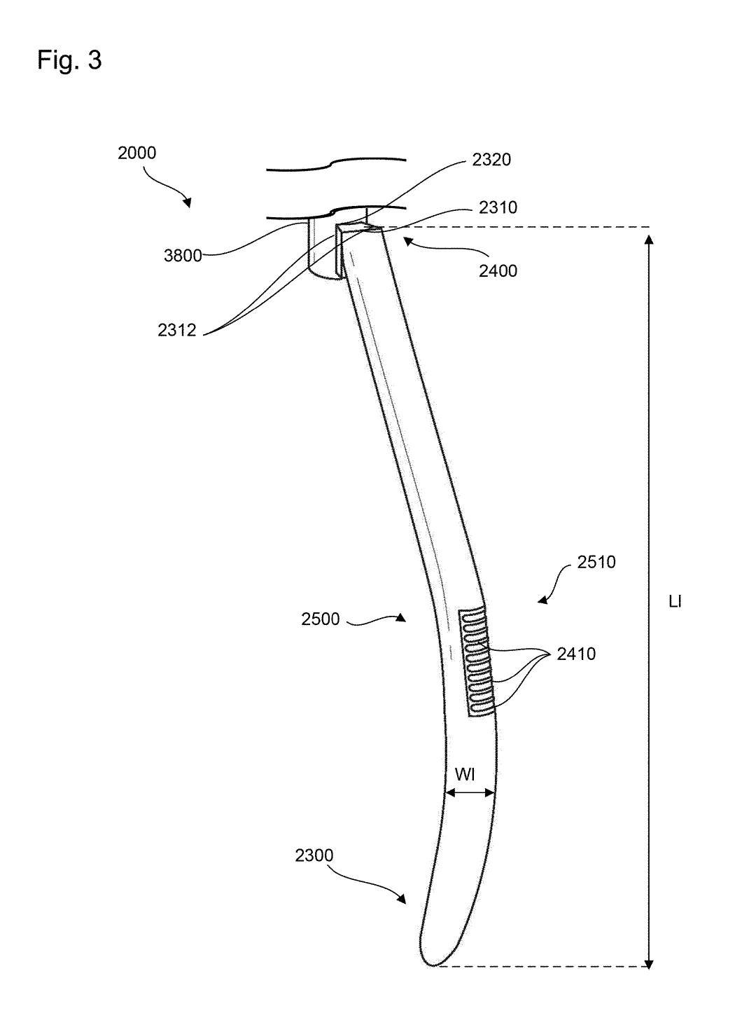

FIG. 3 is a perspective view of a leg of the plurality of legs 2000 of the blood flow diversion device 1000, according to certain aspects of the disclosure.

Each leg of the plurality of legs 2000 can include an downstream terminal portion 2300 positioned at the summit portion 2100, a upstream terminal portion 2400 positioned at the base portion 2200, and a central portion 2500 that connects the downstream terminal portion 2300 with the upstream terminal portion 2400.

The downstream terminal portion 2300 can be narrow and form a tip, the upstream terminal portion 2400 can be large and have a rounded end to avoid damage to the artery internal surface 124, and the central portion 2500 can widen from the downstream terminal portion 2300 to the upstream terminal portion 2400 and have a arched profile to be substantially tangent to the artery internal surface 124 of the parent artery 120, see FIG. 1.

The downstream terminal portion 2300 can include a wedge 2310 and a groove 2320 on the wedge 2310 positioned substantially vertically to receive the branch support 3800 and provide affixing between the plurality of branches 3000 and the plurality of legs 2000.

The wedge 2310 can be defined by a pair of lateral side walls 2312 that radially protrudes from the downstream terminal portion 2300 to connect the groove 2320, wherein the groove 2320 is affixed to the branch support 3800.

The central portion 2500 can include a flatten portion 2510 and/or topologic elements 2410, e.g. plurality of ridges, edges, grooves, and/or protrusions, to provide gripping between the blood flow diversion device 1000 and the artery internal surface 124 of the parent artery 120. Suitable dimensions for the plurality of legs 2000 can be dimensions corresponding to anatomic and/or physiological dimensions, e.g. internal diameter of the parent artery 120, blood pressure, patient age and/or physical conditions. Particularly, each leg 2000 can have a leg length L1, and a leg width, W1 sufficiently large to anchor the blood flow diversion device 1000 onto the parent artery 120 but sufficiently small to enable blood passage towards the children arteries 130. For example, the leg length L1 can be between 0.01 mm and 50.00 mm, and preferably between 0.10 mm and 10.00 mm, the leg width W1 can be between 0.001 mm and 10.000 mm, and preferably between 0.005 mm and 1.000 mm.

Suitable materials for the plurality of legs 2000 can be materials providing biomedical properties, e.g. corrosion resistance, immune system reactivity, and/or biocompatibility, as well as mechanical properties, e.g. elasticity, strength, and or hardness. Particularly, the elasticity of the plurality of the legs 2000 should be sufficiently strong to anchor the blood flow diversion device 1000 onto the parent artery 120 but sufficiently weak to not perforate the parent artery 120.

For example, suitable materials for the plurality legs 2000 can be acrylics, vinyls, nylons, polyurethanes, polycarbonates, poly amides, polysulfones, polyethyleneterephthalate, poly lactic acid, polyglycolic acid, polydimethylsiloxanes, and polyetheretherketones, metals, ceramics, glass, silica, and sapphire. Suitable acrylics can include methyl acrylate, methyl methacrylate, hydroxyethyl methacrylate, hydroxyethyl acrylate, acrylic acid, methacrylic acid, glyceryl acrylate, glyceryl methacrylate, methacrylamide, and acrylamide. Suitable vinyls can include ethylene, propylene, polypropylene, styrene, polystyrene, vinyl chloride, vinyl acetate, vinyl pyrrolidone, and vinylidene difluoride. Suitable nylons can include polycaprolactam, polylauryl lactam, polyhexamethylene adipamide, and polyhexamethylene dodecanediamide. Suitable metals can be titanium, nickel titanium, stainless steel, cobalt chromium, gold, silver, copper, and platinum and their alloys. Suitable ceramics can include silicons such as silicon nitride and silicon carbide, zirconia, and alumina.

Materials of particular interest can include stainless steel, cobalt-chromium, titanium, and nickel-titanium.

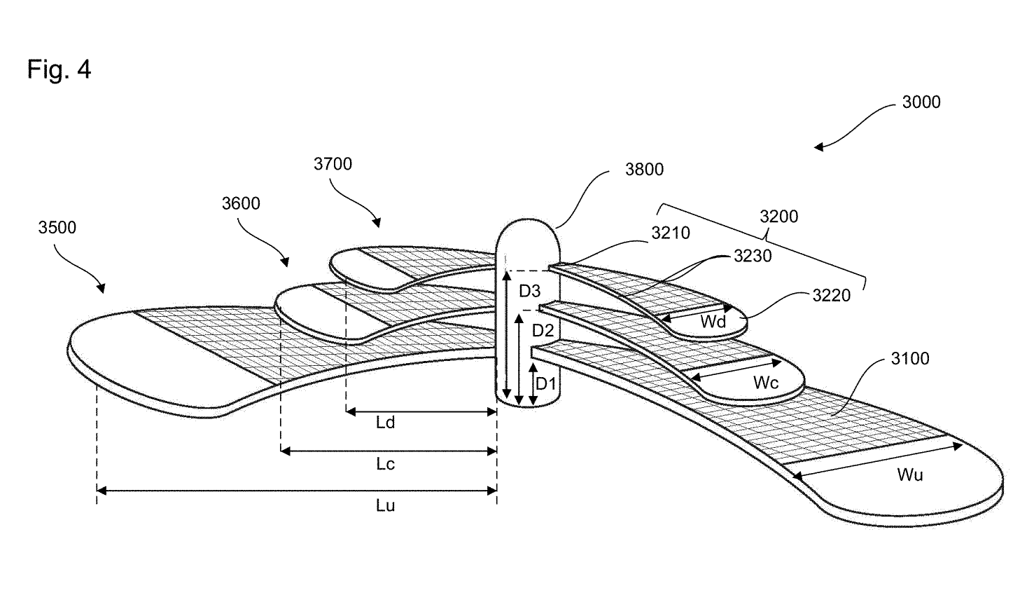

FIG. 4 is a perspective view of the plurality of branches 3000 of the blood flow diversion device 1000, according to certain aspects of the disclosure.

The plurality of branches 3000 can be affixed along the branch support 3800 that is supported by the summit portion 2100 of the the plurality of legs 2000 and can extend radially from the branch support 3800 to cover the aneurysm entrance 112 and expand within the aneurysm 110.

The plurality of branches 3000 can include a plurality of upstream branches 3500 positioned at a first distance D1 downstream from the summit portion 2100 of the plurality of legs 2000, a plurality of central branches 3600 positioned downstream from the plurality of upstream branches 3500 at a second distance D2 from the summit portion 2100, and a plurality of downstream branches 3700 positioned downstream from the plurality of central branches 3600 at a third distance D3 from the summit portion 2100.

The first distance D1, the second distance D2, and the third distance D3 can be determined to ensure that the central branches 3600 and the upstream branches 3500 stay in the vicinity of the aneurysm entrance 112, while the downstream branches 3700 can extend sufficiently downstream from the aneurysm entrance 112 in the internal volume of the aneurysm 110. The first distance D1, the second distance D2, and the third distance D3 can be determined based on a patient morphology such as an artery diameter Da1 and/or an aneurysm diameter Da2, see FIG. 1. For example, the first distance D1 can be between 5% and 95%, and preferably between 20% and 60% of the artery diameter Da1, the second distance D2 can be between 1% and 25%, and preferably between 5% and 20% of the aneurysm diameter Da2, and the third distance D3 can be between 25% and 85%, and preferably between 30% and 75% of the aneurysm diameter Da2.

The branch support 3800 can have a cylindrical shape that can be inserted into the groove 2320 of the wedge 2310 to affix the plurality of branches 3000 to the plurality of legs 2000.

When the blood flow diversion device 1000 is inserted in the artery bifurcation 100 the plurality of upstream branches 3500 can lie on an aneurysm upstream portion 114 of the aneurysm entrance 112, the plurality of central branches 3600 can lie on an aneurysm downstream portion 116 of the aneurysm entrance 112, and the plurality of downstream branches 3700 can extend within an aneurysm internal volume 118 of the aneurysm 110, see FIG. 1.

The plurality of upstream branches 3500, central branches 3600, and downstream branches 3700 can have similar shape but with different dimensions due to different functions and positionings respective to the aneurysm 110. For example, each upstream branch of the plurality of upstream branches 3500 can be characterized by a first length Ld, and a first width Wd, each central branch of the plurality of central branches 3600 can be characterized by a second length Lc and a second width Wc, and each downstream branch of the plurality of downstream branches 3700 can be characterized by a third length Lu and a third width Wu.

The third length and width Lu, Wu can be larger than the first length and width Ld, Wd to have the plurality of upstream branches 3500 lying on the aneurysm upstream portion 114 of the aneurysm entrance 112 and the plurality downstream branches 3700 extending within the aneurysm internal volume 118 of the aneurysm 110, as illustrated in FIG. 1.

The second length and width Lc, Wc can be smaller than the third length and width Ld, Wd but bigger than the first length and width Lu, Wu to have the plurality of central branches 3600 lying on the aneurysm downstream portion 116 the aneurysm entrance 112.

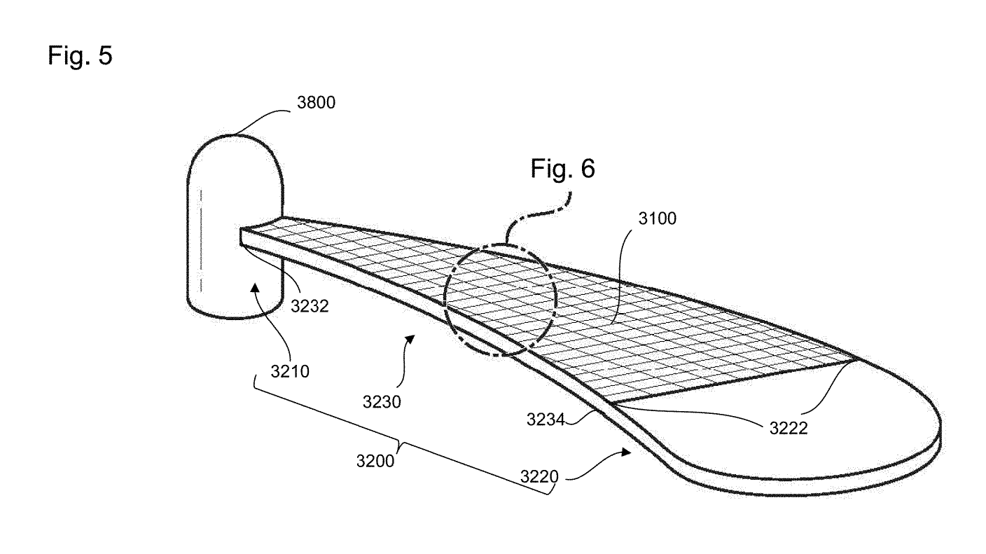

FIG. 5 is a perspective view of a branch of the plurality of branches 3000 of the blood flow diversion device 1000, according to certain aspects of the disclosure.

Each branch of the plurality of branches 3000 can include a branch frame 3200, and a frame mesh 3100 supported by the branch frame 3200.

The branch frame 3200 can include a frame base 3210 that is affixed to the branch support 3800, a frame tip 3220 that is let free in the blood flow, and a pair of arms 3230 that connects the frame base 3210 to the frame tip 3220.

The frame tip 3220 can be a semi-circular plate that harvests hydraulic pressures exerted by the blood flow to deflect the branch frame 3200 and have the branch frame 3200 pushed against the aneurysm entrance 112.

The pair of arms 3230 can have arm anterior ends 3232 that are affixed to the branch support 3800 and arm posterior ends 3234 that are affixed apart from each other on lateral extremities 3222 of the frame tip 3220.

The frame mesh 3100 can stretch between the arm anterior ends 3232 and the arm posterior ends 3234 and be supported by the pair of arms 3230 and the frame tip 3220.

Suitable materials for the branch frame 3200 can be materials providing biomedical properties, e.g. corrosion resistance, immune system reactivity, and/or biocompatibility, as well as flexibility to match shapes of the aneurysm upstream portion 114 and/or the aneurysm downstream portion 116.

For example, suitable materials for the branch frame 3200 can be acrylics, vinyls, nylons, polyurethanes, polycarbonates, poly amides, polysulfones, poly(ethylene terephthalate), poly lactic acid, polyglycolic acid, polydimethylsiloxanes, and polyetheretherketones, metals, ceramics, glass, silica, and sapphire. Suitable acrylics can include methyl acrylate, methyl methacrylate, hydroxyethyl methacrylate, hydroxyethyl acrylate, acrylic acid, methacrylic acid, glyceryl acrylate, glyceryl methacrylate, methacrylamide, and acrylamide. Suitable vinyls can include ethylene, propylene, polypropylene, styrene, polystyrene, vinyl chloride, vinyl acetate, vinyl pyrrolidone, and vinylidene difluoride. Suitable nylons can include polycaprolactam, polylauryl lactam, polyhexamethylene adipamide, and polyhexamethylene dodecanediamide. Suitable metals can be titanium, nickel titanium, stainless steel, cobalt chromium, gold, silver, copper, and platinum and their alloys. Suitable ceramics can include silicons such as silicon nitride and silicon carbide, zirconia, and alumina.

Materials of particular interest can include stainless steel, cobalt-chromium, titanium, and nickel-titanium.

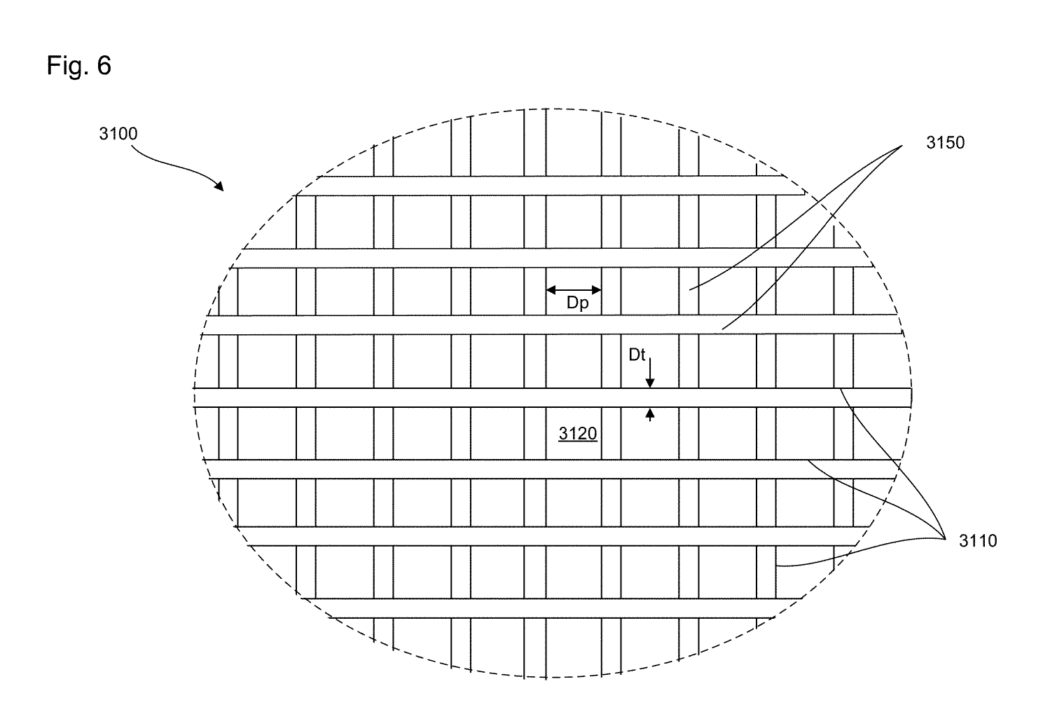

FIG. 6 is a close up view of the frame mesh 3100 of the branch 3000, according to certain aspects of the disclosure.

The frame mesh 3100 can include a plurality of threads 3110 intercrossing each other to provide a plurality of pores 3120 that slow down blood passage, promote blood coagulation, and generate thrombosis.

The plurality of pores 3120 and the plurality of threads 3110 have relative dimensions to ensure that there will be a predetermined resistance to blood flow through the frame mesh 3100 which in turn ensures that there will be overgrowth of endothelial cells around the plurality of threads 3110 so as to induce coagulation and the generation of thrombosis.

For example, each pore of the plurality of pores 3120 can be characterized by a pore spacing Dp and each thread of the plurality of threads 3110 can be characterized by a thread diameter Dt, wherein the ratio between the pore spacing Dp and the thread diameter Dt is between least 0.1 and 100.0, preferably between 0.5 and 10.0, and more preferably between 1.0 and 5.0. The pore spacing Dp can be smaller than 20 microns, preferably smaller than 10 microns, and more preferably smaller than 2 microns.

Alternatively, the thread diameter Dt can be larger than a predetermined threshold, wherein the predetermined threshold can be between 0.1 micron and 2 microns, preferably between 0.5 micron an 1.5 microns, and more preferably between 0.75 micron and 1 micron.

In addition, a biocompatible coating 3150 can be applied on the frame mesh 3100 to enhance coagulation around the plurality of threads 3110 and/or adherence of the branch 3000 onto the aneurysm upstream portion 114 and/or the aneurysm downstream portion 116. For example, the biocompatible coating 3150 can be a coating which encourage fibroblast ingrowth, e.g. Transforming Growth Factor beta (TGF-β), or a tissue irritant, or endothelial ingrowth, e.g. Vascular Endothelial Growth Factor (VEGF).

The biocompatible coating 3150 can have a predetermined thickness Tc sufficiently small to not affect mechanical properties of the blood flow diversion device 1000 but sufficiently large to enhance coagulation and/or adherence. For example, the predetermined thickness Tc of biocompatible coating 3150 can be between 0.01 micron and 10.00 microns, preferably between 0.10 micron and 5.00 microns, and more preferably between 0.50 micron and 2.00 microns.

Alternatively, other and/or different coatings may be applied on other and/or different portions of the blood flow diversion device 1000, e.g. the plurality of legs 2000 and/or the plurality of branches 3000, to provide other and/or different functions.

For example, a treatment coating can be applied on the frame tip 3220 of the plurality of branches 3000 to administer a pharmaceutically active material to the artery bifurcation 100 and/or the aneurysm 110 to enhance the diversion of the blood flow towards the children arteries 130. The pharmaceutical active material can be a blood thinner such as aspirin, heparin, and/or orwarfarin.

In another example, a tissue reactive coating can be applied on the upstream terminal portions 2400 of the plurality of legs 2000 to enhance the anchorage of the blood flow diversion device 1000 on the parent artery 120. The tissue reactive coating may include VEGF and nanotube matrices to promote growth tissue between the blood flow diversion device 1000 and the parent artery 120.

The foregoing discussion discloses and describes merely exemplary embodiments of an object of the present disclosure. As will be understood by those skilled in the art, an object of the present disclosure may be embodied in other specific forms without departing from the spirit or essential characteristics thereof. Accordingly, the present disclosure is intended to be illustrative, but not limiting of the scope of an object of the present disclosure as well as the claims.

Numerous modifications and variations on the present disclosure are possible in light of the above teachings. It is therefore to be understood that within the scope of the appended claims, the disclosure may be practiced otherwise than as specifically described herein.

What is claimed is:

1. A blood flow diversion device to treat an aneurysm at a bifurcation artery, the blood flow diversion device comprising:
a plurality of legs configured to anchor the blood flow diversion device onto a parent artery of the bifurcation artery;
a branch support affixed to the plurality of legs;
a plurality of branches that extends from the branch support, the plurality of branches including:
a plurality of upstream branches positioned above the plurality of legs and configured to lie on a upstream portion of an aneurysm entrance of the aneurysm and divert blood flow towards other arteries of the bifurcation artery, and
a plurality of downstream branches positioned above the plurality of upstream branches and configured to extend within an internal volume of the aneurysm and promote thrombosis formation in the internal volume.

2. The blood flow diversion device of claim 1, wherein the plurality of branches include a plurality of central branches positioned between the plurality of upstream branches and the plurality of downstream branches and configured to lie on an downstream portion of the aneurysm entrance to impede the blood flow inside the aneurysm.

3. The blood flow diversion device of claim 2, wherein the plurality of upstream branches each have a first length, the plurality of central branches each have a second length smaller than the first length, and the plurality of downstream branches each have a third length smaller than the second length.

4. The blood flow diversion device of claim 1, wherein each branch of the plurality of branches includes a frame mesh to impede the blood flow, and a frame branch to support the frame mesh.

5. The blood flow diversion device of claim 4, wherein the frame branch includes a pair of frame arms that protrude radially from the branch support to provide support to the frame mesh.

6. The blood flow diversion device of claim 5, wherein the frame branch further includes:
a frame tip affixed to terminal ends of the pair of frame arms, the frame tip being configured to harvest hydraulic pressures exerted by the blood flow and deflect the frame branch onto the aneurysm entrance.

7. The blood flow diversion device of claim 6, wherein the frame mesh can include a plurality of threads intercrossing each other to provide a plurality of pores and inhibit the blood flow.

8. The blood flow diversion device of claim 7, wherein each thread of the plurality of threads has a thread diameter larger than a predetermined threshold to generate overgrowth of endothelial cells around the plurality of threads.

9. The blood flow diversion device of claim 4, wherein the frame mesh includes a biocompatible coating to encourage fibroblast ingrowth and coagulation.

10. A blood flow diversion device to treat an aneurysm at a bifurcation artery, the blood flow diversion device comprising:
a plurality of legs configured to anchor the blood flow diversion device onto a parent artery of the bifurcation artery;
a branch support affixed to the plurality of legs; and
a plurality of branches that extends from the branch support to impede blood flow entering the aneurysm and promote thrombosis.

11. The blood flow diversion device of claim 10, wherein each leg of the plurality of legs includes an downstream portion affixed to the branch support.

12. The blood flow diversion device of claim 10, wherein the downstream portion includes a wedge to provide affixing between each leg and the branch support.

13. The blood flow diversion device of claim 12, wherein the wedge includes a groove to receive the branch support.

14. The blood flow diversion device of claim 10, wherein each leg of the plurality of legs includes a upstream terminal portion spaced apart and free from other upstream terminal portions of the plurality of legs to extend radially and a central portion to contact an internal surface of the parent artery.

15. The blood flow diversion device of claim 14, wherein the central terminal portion has a flatten portion to contact the internal surface of the parent artery.

16. The blood flow diversion device of claim 15, wherein the flatten portion include topological elements to enhance anchorage between the parent artery and the plurality of legs.

17. The blood flow diversion device of claim 14, wherein the upstream terminal portion includes a tissue reactive coating to enhance anchorage between the parent artery and the plurality of legs.

18. The blood flow diversion device of claim 10, wherein the plurality of branches includes:
a plurality of upstream branches positioned above the plurality of legs to lie on a upstream portion of an aneurysm entrance of the aneurysm and divert the blood flow towards children arteries of the bifurcation artery; and
a plurality of downstream branches positioned above the plurality of upstream branches to extend within an internal volume of the aneurysm and promote thrombosis.

19. The blood flow diversion device of claim 18, wherein the plurality of branches includes a plurality of central branches positioned between the plurality of upstream branches and the plurality of downstream branches to lie on an downstream portion of the aneurysm entrance to impede the blood flow inside the aneurysm.

20. The blood flow diversion device of claim 19, wherein the plurality of upstream branches has a first length, the plurality of central branches has a second length smaller than the first length, and the plurality of downstream branches has a third length smaller than the second length.
